(19) United States
(12) Patent Application Publication
(10) Pub. No.: US 2019/0216991 A1
(43) Pub. Date: Jul. 18, 2019

(54) Pressure Sensing Dressing Interface

(71) Applicant: KCI LICENSING, INC., San Antonio, TX (US)

(72) Inventors: Christopher Brian LOCKE, Bournemouth (GB); James A. LUCKEMEYER, San Antonio, TX (US); Timothy Mark ROBINSON, Shillingstone (GB)

(73) Assignee: KCI Licensing, Inc., San Antonio, TX (US)

(21) Appl. No.: 16/301,692

(22) PCT Filed: Apr. 17, 2017
(86) PCT No.: PCT/US2017/027975

§ 371 Date: Nov. 14, 2018

Related U.S. Application Data
(60) Provisional application No. 62/346,010, filed on Jun. 6, 2016.

(51) Int. Cl. A61M 1/00; A61L 31/08; A61L 31/14; A61M 39/10; G01L 21/00

(52) U.S. Cl. CPC A61M 1/0088; A61L 31/088; A61L 31/146; A61M 2039/1022; G01L 21/00; A61M 2205/3317; A61M 2205/3344; A61M 39/10

(57) ABSTRACT

Methods, apparatuses, and systems for determining a pressure in a sealed therapeutic environment provided by a dressing are described. The system can include a tissue interface configured to be positioned adjacent a tissue site. At least a portion of the tissue interface is electrically conductive. The system also includes a sealing member configured to be disposed over the tissue interface to form the sealed therapeutic environment. The system includes a dressing interface configured to fluidly connect the sealed therapeutic environment with a therapy unit. The dressing interface is further configured to be electrically coupled to the tissue interface and the therapy unit.

### RELATED APPLICATION

This application claims the benefit, under 35 USC 119(e), of the filing of U.S. Provisional Patent Application No. 62/346,010, entitled “Pressure Sensing Dressing Interface,” filed Jun. 6, 2016, which is incorporated herein by reference for all purposes.

### TECHNICAL FIELD

The invention set forth in the appended claims relates generally to tissue treatment systems and more particularly, but without limitation, to pressure detection in a sealed therapeutic environment.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound can be washed out with a stream of liquid solution, or a cavity can be washed out using a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, the cost and complexity of therapy can be a limiting factor in its application, and the development and operation of therapy systems, components, and processes continues to present significant benefits to healthcare providers and patients.

### BRIEF SUMMARY

New and useful systems, apparatuses, and methods for determining a pressure in a sealed therapeutic environment are set forth in the appended claims. Illustrative embodiments are also provided to enable a person skilled in the art to make and use the claimed subject matter.

For example, in some embodiments, a system and method for using the system to determine a pressure in a sealed therapeutic environment is described that can determine the pressure without the use of an expensive wound based pressure sensor.

More generally, a system for determining a pressure in a sealed therapeutic environment provided by a dressing is described. The system can include a tissue interface configured to be positioned adjacent a tissue site. At least a portion of the tissue interface may be electrically conductive. The system can also include a sealing member configured to be disposed over the tissue interface to form the sealed therapeutic environment. The system includes a dressing interface configured to fluidly connect the sealed therapeutic environment with a therapy unit. The dressing interface can be further configured to be electrically coupled to the tissue interface and the therapy unit.

Alternatively, other example embodiments describe a method for determining a pressure in a sealed space. A tissue interface having a conductive portion can be applied adjacent to a tissue site. The tissue interface can be covered with a sealing member to form the sealed space. A dressing interface having at least two electrodes may be coupled to the sealing member adjacent an aperture in the sealing member so that the at least two electrodes are electrically coupled to the conductive portion of the tissue interface. The at least two electrodes can be electrically coupled to a therapy unit. The dressing interface can be fluidly coupled to the therapy unit. A load voltage across the electrodes can be monitored. In response to a change in the load voltage across the electrodes, a pressure in the sealed space can be determined.

Some example embodiments describe a system for treating a tissue site. The system can include a manifold having a conductive portion capable of conducting an electric current and configured to be positioned adjacent to the tissue site. The system can also include a cover configured to be positioned over the manifold and the tissue site to form a therapeutic environment. A connector may be configured to be fluidly coupled to the therapeutic environment and the manifold to a source of negative pressure. The connector may have two electrodes coupled to the connector and configured to be electrically coupled to the conductive portion of the manifold. The system can also include a therapy unit configured to fluidly couple the source of negative pressure to the connector. The therapy unit can be configured to be electrically coupled to the two electrodes to provide a current to the conductive portion of the manifold. The therapy unit may be further configured to measure a therapy voltage across the two electrodes as a measure of therapy pressure within the therapeutic environment in response to pressure being provided by the source of negative pressure to the therapeutic environment and the manifold.

Another example embodiment describes a method for determining a pressure in a sealed therapeutic environment. An initial voltage across at least two electrodes electrically connected to a tissue interface disposed in the sealed therapeutic environment can be determined. A sealed therapeutic environment voltage can be determined after operation of a therapy unit. If the sealed therapeutic environment voltage is about the same as a therapy voltage, the pressure in the sealed therapeutic environment can be identified as the therapy pressure. If the sealed therapeutic environment voltage is not about the same as a therapy voltage, the pressure in the sealed therapeutic environment can be identified as being not about the therapy pressure.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can determine a pressure in a sealed therapeutic environment in accordance with this specification;

FIG. 2A is a top perspective view of a dressing interface that may be used with the therapy system of FIG. 1;

FIG. 2B is a bottom perspective view of the dressing interface of FIG. 2A;

FIG. 2C is a bottom view of the dressing interface of FIG. 2A;

FIG. 2D is a bottom perspective view of another dressing interface that may be used with the therapy system of FIG. 1;

FIG. 2E is a bottom perspective view of another dressing interface that may be used with the therapy system of FIG. 1;

FIG. 2F is a bottom perspective view of another dressing interface that may be used with the therapy system of FIG. 1;

FIG. 2G is a bottom perspective view of another dressing interface that may be used with the therapy system of FIG. 1;

FIG. 2H is an end view of the dressing interface of FIG. 2A;

FIG. 2I is a sectional view of the dressing interface taken along line 2I-21 of FIG. 2H;

FIG. 3A is a perspective view of a portion of a tube that may be used with the dressing interface of FIG. 2A;

FIG. 3B is a sectional view of the tube of FIG. 3A;

FIG. 3C is a sectional view of another tube that may be used with the dressing interface of FIG. 2A;

FIG. 4A is a sectional view of the dressing and a schematic view of the therapy system of FIG. 1 disposed over a tissue site an illustrating additional details that may be associated with some embodiments;

FIG. 4B is a detail view of a portion of a coupling between a dressing interface and a tube of FIG. 4A;

FIGS. 5A-5B are high-level flow charts illustrating additional details that may be associated with the operation of the therapy system of FIG. 1;

FIG. 6 is a graphical depiction of the relationship between a pressure in the sealed therapeutic environment and a signal provided by the system in an exemplary embodiment of the therapy system of FIG. 1;

FIG. 7 is a graphical depiction of the relationship between a pressure in the sealed therapeutic environment and a signal provided by the system in another exemplary embodiment of the therapy system of FIG. 1; and

FIG. 8 is a graphical depiction of the relationship between a pressure in the sealed therapeutic environment and a signal provided by the system in another exemplary embodiment of the therapy system of FIG. 1.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

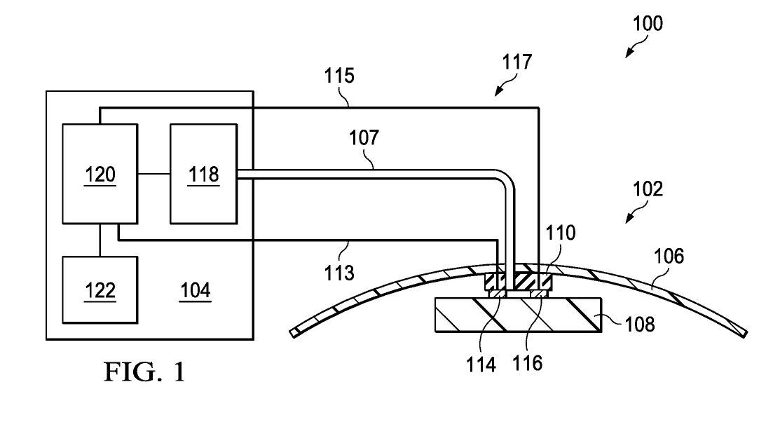

FIG. 1 is a simplified functional block diagram of an example embodiment of a therapy system 100 that can provide negative-pressure therapy in accordance with this specification. The therapy system 100 may include a negative-pressure supply, and may include or be configured to be coupled to a distribution component, such as a dressing. In general, a distribution component may refer to any complementary or ancillary component configured to be fluidly coupled to a negative-pressure supply in a fluid path between a negative-pressure supply and a tissue site. A distribution component is preferably detachable, and may be disposable, reusable, or recyclable. For example, a dressing 102 may be fluidly coupled to a negative-pressure source 104, as illustrated in FIG. 1. A dressing may include a cover, a tissue interface, or both in some embodiments. The dressing 102, for example, may include a sealing member 106 and a tissue interface 108.

In some embodiments, a connector, such as a dressing interface 110 may facilitate coupling the negative-pressure source 104 to the dressing 102. For example, such a dressing interface may be similar to a T.R.A.C.® Pad or Sensa T.R.A.C.® Pad available from Kinetic Concepts, Inc. of San Antonio, Tex. The therapy system 100 may optionally include a fluid container coupled to the dressing 102 and to the negative-pressure source 104.

Components may be fluidly coupled to each other to provide a path for transferring fluids (i.e., liquid and/or gas) between the components. For example, components may be fluidly coupled through a fluid conductor, such as a tube 107. A “tube,” as used herein, broadly includes a tube, pipe, hose, conduit, or other structure with one or more lumina adapted to convey a fluid between two ends. Typically, a tube is an elongated, cylindrical structure with some flexibility, but the geometry and rigidity may vary. In some embodiments, components may also be coupled by virtue of physical proximity, being integral to a single structure, or being formed from the same piece of material. Moreover, some fluid conductors may be molded into or otherwise integrally combined with other components. Coupling may also include mechanical, thermal, electrical, or chemical coupling (such as a chemical bond) in some contexts. For example, the tube 107 may mechanically and fluidly couple the dressing 102 to the negative-pressure source 104 in some embodiments.

In general, components of the therapy system 100 may be coupled directly or indirectly. For example, the negative-pressure source 104 may be directly coupled to the tube 107, and may be indirectly coupled to the dressing 102 through the tube 107 and the dressing interface 110.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically refers to a position in a fluid path relatively closer to a source of negative pressure or further away from a source of positive pressure. Conversely, the term “upstream” refers to a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” generally refers to a pressure less than a local ambient pressure, such as the ambient pressure in a local environment external to a sealed therapeutic environment provided by the dressing 102. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between about −5 mm Hg (−667 Pa) and about −500 mm Hg (−66.7 kPa). Common therapeutic ranges are between about −75 mm Hg (−9.9 kPa) and about −300 mm Hg (−39.9 kPa).

A negative-pressure supply, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 104 may be combined with a controller and other components into a therapy unit. A negative-pressure supply may also have one or more supply ports configured to facilitate coupling and de-coupling the negative-pressure supply to one or more distribution components.

In some embodiments, the negative-pressure source 104 may include a pump 118, a controller 120, and an input-output device 122. The controller 120 may be electrically coupled to the pump 118 and the input-output device 122. In some embodiments, the controller 120 may also be electrically coupled to a sensing circuit 117 that includes a first conductive pathway 113, a second conductive pathway 115, a first electrode 114, and a second electrode 116.

A therapy device, such as the negative-pressure source 104, may include a user interface, such as the input-output device 122. A user interface may be a device configured to allow communication between a controller and an environment external to a therapy device. An external environment may include an operator or a computer system configured to interface with a therapy device, for example. In some embodiments, a user interface may receive a signal from a controller and present the signal in a manner that may be understood by an external environment. In some embodiments, a user interface may receive signals from an external environment and, in response, send signals to a controller.

In some embodiments, a user interface may be a graphical user interface, a touchscreen, or one or more motion tracking devices. A user interface may also include one or more display screens, such as a liquid crystal display (“LCD”), lighting devices, such as light emitting diodes (“LED”) of various colors, and audible indicators, such as a whistle or tone generator, configured to emit a sound that may be heard by an operator. A user interface may further include one or more devices, such as knobs, buttons, keyboards, remotes, touchscreens, remote devices, such as smartphones, flexible displays, ports that may be configured to receive a discrete or continuous signal from another device, or other similar devices; these devices may be configured to permit the external environment to interact with the user interface. A user interface may permit an external environment to select a therapy to be performed with a therapy device. In some embodiments, a user interface may display information for an external environment such as a duration of therapy, a type of therapy, an amount of negative pressure being supplied, an amount of instillation solution being provided, a fluid level of a container, or a fluid level of a cartridge, for example.

A therapy device, such as the negative-pressure source 104, may include one or more valves. Generally, a valve may be configured to selectively permit fluid flow through the valve. A valve may be a ball valve, a gate valve, a butterfly valve, or other valve type that may be operated to control fluid flow through the valve. Generally, a valve may include a valve body having a flow passage, a valve member disposed in the flow passage and operable to selectively block the flow passage, and an actuator configured to operate the valve member. An actuator may be configured to position the valve member in a closed position, preventing fluid flow through the flow passage of the valve; an open position, permitting fluid flow through the fluid passage of the valve; or a metering position, permitting fluid flow through the flow passage of the valve at a selected flow rate. In some embodiments, the actuator may be a mechanical actuator configured to be operated by an operator or user. In some embodiments, the actuator may be an electromechanical actuator configured to be operated in response to the receipt of a signal input. For example, the actuator may include an electrical motor configured to receive a signal from a controller. In response to the signal, the electrical motor of the actuator may move the valve member of the valve. In some embodiments, a valve may be configured to selectively permit fluid communication between the negative-pressure source 104 and the dressing 102. In some embodiments, the negative-pressure source 104 may include valves configured to permit venting of the dressing 102 by allowing ambient air to flow through the negative-pressure source 104 to the dressing 102. In other embodiments, the negative-pressure source 104 may include one or more valves operable to permit the negative-pressure source 104 to supply an instillation fluid to a tissue site.

A therapy device may include one or more controllers, such as the controller 120, electrically coupled to components of the therapy device, such as a valve, a flow meter, a sensor, a user interface, or a pump, for example, to control operation of the same. As used herein, communicative coupling may refer to a coupling between components that permits the transmission of signals between the components. In some embodiments, the signals may be discrete or continuous signals. A discrete signal may be a signal representing a value at a particular instance in a time period. A plurality of discrete signals may be used to represent a changing value over a time period. A continuous signal may be a signal that provides a value for each instance in a time period. The signals may also be analog signals or digital signals. An analog signal may be a continuous signal that includes a time varying feature that represents another time varying quantity. A digital signal may be a signal composed of a sequence of discrete values. A signal can include a variable parameter that contains information and by which information is transmitted in an electronic system or circuit. A signal can be a voltage source in which the amplitude, frequency, and waveform can be varied. Communicative coupling can also include coupling between one or more components that permits an electric current to flow between the components.

In some embodiments, communicative coupling between a controller and other devices may be one-way communication. In one-way communication, signals may only be sent in one direction. For example, a sensor may generate a signal that may be communicated to a controller, but the controller may not be capable of sending a signal to the sensor. In some embodiments, communicative coupling between a controller and another device may be two-way communication. In two-way communication, signals may be sent in both directions. For example, a controller and a user interface may be electrically coupled so that the controller may send and receive signals from the user interface. Similarly, a user interface may send and receive signals from a controller. In some embodiments, signal transmission between a controller and another device may be referred to as the controller operating the device. For example, interaction between a controller and a valve may be referred to as the controller: operating the valve; placing the valve in an open position, a closed position, or a metering position; and opening the valve, closing the valve, or metering the valve.

A controller may be a computing device or system, such as a programmable logic controller (PLC), or a data processing system, for example. In some embodiments, a controller may be configured to receive input from one or more devices, such as a user interface, a sensor, or a flow meter, for example. In some embodiments, a controller may receive input, such as an electrical signal, from an alternative source, such as through an electrical port, for example. Other examples input examples can include wireless or optical signals.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code in order to reduce the number of times code is retrieved from bulk storage during execution.

A PLC may be a digital computer configured to receive one or more inputs and send one or more outputs in response to the one or more inputs. A PLC may include a non-volatile memory configured to store programs or operational instructions. In some embodiments, the non-volatile memory may be operationally coupled to a battery-back up so that the non-volatile memory retains the programs or operational instructions if the PLC otherwise loses power. In some embodiments, a PLC may be configured to receive discrete signals and continuous signals and produce discrete and continuous signals in response. A PLC can also be configured to include non-volatile flash memory capable of storing configuration settings that can be retrieved after a power-cycle of the device.

A controller may also include a power source. A power source may be a source of electrical energy, such as a battery, or an inverter electrically coupled to a mains electricity supply. The power source may be capable of supplying electrical energy to other components in response to a signal from the controller.

The tissue interface 108 can be generally adapted to contact a tissue site. The tissue interface 108 may be partially or fully in contact with the tissue site. If the tissue site is a wound, for example, the tissue interface 108 may partially or completely fill the wound, or may be placed over the wound to create a cavity over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites. Moreover, any or all of the surfaces of the tissue interface 108 may have projections or an uneven, coarse, or jagged profile that can induce strains and stresses on a tissue site, which can promote granulation at the tissue site.

In some embodiments, the tissue interface 108 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 108 may be a foam having pore sizes in a range of about 400 microns to about 600 microns. The tensile strength of the tissue interface 108 may also vary according to needs of a prescribed therapy. For example, the tensile strength of a foam may be increased for instillation of topical treatment solutions. In one non-limiting example, the tissue interface 108 may be similar to an open-cell, reticulated polyurethane foam such as GranuFoam® dressing or VeraFlo® foam, both available from Kinetic Concepts, Inc. of San Antonio, Tex.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

The tissue interface 108 may also be electrically conductive. For example, the tissue interface 108 may be an open-cell reticulated foam having a thin layer of silver coated onto the foam. The reticulated, open-cell structure of the foam may be maintained after the coating process. For example, following the application of a silver coating, the foam may have pore sizes in the range of about 400 microns to about 600 microns. In some embodiments, the silver coating may have a thickness of about 1 micron to about 10 microns and, in particular, about 3 microns. The coating of silver may extend through the open-cell reticulated foam of the tissue interface 108 so that substantially all surfaces of the tissue interface 108 may be coated. In other embodiments, the silver coating may only be applied to a portion of the tissue interface 108 or to a surface of the tissue interface 108 to form a conductive portion. The silver coating may be 99.9% pure metallic silver that is bonded to the tissue interface 108. The silver coating may have an electrical resistivity of about 1.59×10⁻⁸ ohm meters at 20 degrees Celsius. In other embodiments, the tissue interface 108 may be coated with copper, gold, or other metallic materials. The tissue interface 108 may also be formed from conductive materials such as a poly aniline foam, a poly acetylene foam, or a poly polystyrene sulphonate foam. In some embodiments, the tissue interface 108 may be V.A.C. GranuFoam Silver® Dressing available from KCl, Inc. In some embodiments, the silver coating may provide antibacterial properties.

In some embodiments, the sealing member 106 may provide a bacterial barrier and protection from physical trauma. The sealing member 106 may also be constructed from a material that can reduce evaporative losses and provide a fluid seal between two components or two environments, such as between a therapeutic environment and a local external environment. The sealing member 106 may be, for example, an elastomeric film or membrane that can provide a seal adequate to maintain a negative pressure at a tissue site for a given negative-pressure source. The sealing member 106 may have a high moisture-vapor transmission rate (MVTR) in some applications. For example, the MVTR may be at least about 300 grams per meter squared per twenty-four hours. In some embodiments, the sealing member 106 may be a polymer drape, such as a polyurethane film, that is permeable to water vapor but impermeable to liquid. Such drapes typically have a thickness in the range of about 25 microns to about 50 microns. For permeable materials, the permeability generally should be low enough that a desired negative pressure may be maintained.

An attachment device may be used to attach the sealing member 106 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the sealing member 106 may be coated with an acrylic adhesive having a coating weight between about 25 grams per square meter (gsm) and about 65 gsm. Thicker adhesives, or combinations of adhesives, may be applied in some embodiments to improve the seal and reduce leaks. For example, an attachment device may comprise a first layer of adhesive disposed on a film layer and a second layer of adhesive disposed on the first layer of adhesive. The second layer of adhesive may include one or more apertures permitting the first layer of adhesive to pass through the second layer of adhesive. The first layer of adhesive may have a bond strength that is greater than a bond strength of the second layer of adhesive. Other example embodiments of an attachment device may include a double-sided tape, paste, hydrocolloid, hydrogel, silicone gel, or organogel.

In operation, the tissue interface 108 may be placed within, over, on, or otherwise proximate to a tissue site. The sealing member 106 may be placed over the tissue interface 108 and sealed to an attachment surface near the tissue site. For example, the sealing member 106 may be sealed to undamaged epidermis peripheral to a tissue site. Thus, the dressing 102 can provide a sealed therapeutic environment proximate to a tissue site, substantially isolated from the external environment, and the negative-pressure source 104 can reduce the pressure in the sealed therapeutic environment. Negative pressure applied across the tissue site through the tissue interface 108 in the sealed therapeutic environment can induce macrostrain and microstrain in the tissue site, as well as remove exudates and other fluids from the tissue site, which can be collected in a container.

Negative-pressure therapy systems often measure pressure at a tissue site pneumatically. For example, a negative-pressure source may include a pneumatic sensor fluidly coupled between a negative-pressure outlet of a negative-pressure source and a tissue site. The pneumatic sensor may measure a pressure and represent a pressure at the tissue site with the pressure measured at the negative-pressure source. In some cases, measuring the pressure at the negative-pressure outlet of the negative-pressure source may not reflect the pressure at the tissue site. For example, blockages in a tube fluidly coupling a negative-pressure outlet and a tissue site may prevent correlation between the pressure at the tissue site and the pressure at the negative-pressure outlet. Similarly, leaks in a therapy system between a negative-pressure source and a tissue site may cause a pressure at a negative-pressure outlet to differ from the pressure at the tissue site. If a pressure is pneumatically measured at a canister or container, blockages or leaks between the canister and a tissue site may also cause a therapy system to misrepresent the pressure at the tissue site.

Some therapy systems pneumatically measure a pressure at a tissue site by measuring a pressure in a tube fluidly coupled to the tissue site independently of a negative-pressure outlet of a negative-pressure source. Generally, a fluid volume at a tissue site is connected by a fluid column to a pneumatic sensor. The fluid column is only connected to a negative-pressure source through the tissue site. Consequently, changes in pressure at the tissue site caused by activation of the negative-pressure source may affect the fluid column, causing a change in pressure of the fluid column. The pneumatic sensor can measure the pressure of the fluid column to determine the pressure at the tissue site, solving many of the problems associated with measuring pressure at a negative-pressure outlet of a negative-pressure source. Increased sensitivity can be obtained by using a fluid column that is relatively small volume compared to a volume of the sealed therapeutic environment.

Maintaining a relatively small volume of a fluid column compared to the volume of the sealed therapeutic environment can be accomplished using multiple tubes. For example, a negative-pressure source can be coupled to a tissue site with a delivery tube and a pressure-sensing tube. A delivery tube may have a single lumen having a diameter facilitating the flow of fluids, negative-pressure, and other debris away from a tissue site. A pressure-sensing tube may have a lumen with a diameter an order of magnitude smaller in dimension than a diameter of the delivery tube. The pressure-sensing tube can be used exclusively for the purpose of measuring a pressure at a tissue site. Additional pressure-sensing tubes may be coupled to a tissue site to provide redundancy in the event any one of the pressure-sensing tubes becomes blocked. However, using multiple tubes can complicate use of a therapy system and lead to patient discomfort.

Some therapy systems use a multi-lumen conduit or multi-lumen tube with dedicated delivery lumens and pressure-sensing lumens. For example, a multi-lumen tube may have a central lumen and one or more peripheral lumens. The central lumen may provide a path for the communication of negative pressure from a source of negative pressure to a tissue site. In addition, the central lumen may provide a pathway for liquids and other materials from the tissue site to move away from the tissue site and into, for example, a canister or container. Each of the peripheral lumens may have a diameter that is less than a diameter of the central lumen. In some embodiments, each of the peripheral lumens may have a diameter that is less than half of the diameter of the central lumen. With a multi-lumen conduit, a blockage in the negative-pressure delivery conduit does not compromise pressure sensing, and a blockage in a pressure-sensing lumen does not compromise negative-pressure delivery. However, a blockage or leak at the connector or an improperly positioned connector may inhibit the operation of the system. In addition, a multi-lumen conduit may require a special dressing interface configured to fluidly couple both the primary lumen and the peripheral lumens to a tissue site.

Other systems may monitor pressure at a tissue sit by positioning an electronic pressure sensor at the tissue site. Electronic pressure sensors may rely on a transducer that can generate a signal as a function of a pressure applied to the transducer. The electronic pressure sensor can be electrically coupled to a therapy system. However, electronic pressure sensors can be prohibitively expensive. An electronic pressure sensor can also be prone to fouling in the presence of wound fluids at a tissue site, causing the electronic pressure sensor to report inaccurate pressures. Electronic pressure sensors can also be complicated to position and can require extensive training for users to ensure proper placement. Furthermore, because the electronic pressure sensor is disposed at the tissue site and exposed to fluids from the tissue site, the electronic pressure sensor must be discarded after a single use, further increasing costs of therapy systems employing such sensors.

The therapy system 100 can address many of the issues associated with measuring pressure at a tissue site describe above. The therapy system 100 may include a pressure sensing mechanism that can measure pressure at a tissue site using a tissue interface such as, for example, the tissue interface 108. The tissue interface may have a conductive portion that is electrically conductive, and the therapy system 100 can apply an electrical current to the conductive portion to induce a voltage drop across the tissue interface that can be correlated to the pressure at the tissue site. For example, the tissue interface may comprise a foam having a conductive portion. If a negative pressure is applied to the therapeutic environment, the tissue interface deforms or compresses. As the tissue interface compresses, the resistance of the conductive portion decreases, i.e., the conductivity increases, causing the voltage drop across the tissue interface to decrease proportionally. As a result, the voltage drop across the tissue interface can be measured as a measure of the negative pressure at the tissue site. A therapy unit, such as the negative-pressure source 104, can be configured to apply electrical current to the conductive portion of the tissue interface, to measure the induced voltage drop, and to compute the corresponding pressure at the tissue site.

In some embodiments, the dressing interface 110 may include electrical contacts, such as the first electrode 114 and the second electrode 116. The first electrode 114 and the second electrode 116 may be electrically coupled to a therapy device, such as the negative-pressure source 104. In some embodiments, the first electrode 114 may be electrically coupled to the controller 120 of the negative-pressure source 104 through the first conductive pathway 113. The second electrode 116 may be electrically coupled to the controller 120 of the negative-pressure source 104 through the second conductive pathway 115. Both the first electrode 114 and the second electrode 116 may contact the tissue interface 108 to complete the sensing circuit 117. During operation of the negative-pressure source 104, the controller 120 may supply a current to the sensing circuit 117 and determine a resistance of the sensing circuit 117. As fluid is drawn from the sealed therapeutic environment, the resistance of the tissue interface 108 may change, and the sensing circuit 117 may communicate the change in resistance to the negative-pressure source 104. The change in resistance communicated by the sensing circuit 117 can be correlated to a change in pressure at a tissue site where the tissue interface 108 is disposed.

The first electrode 114 and the second electrode 116 may be coupled to the dressing interface 110 and positioned so that, if the dressing interface 110 is coupled to the dressing 102, the first electrode 114 and the second electrode 116 may be electrically coupled to the tissue interface 108 and complete the sensing circuit 117. The first electrode 114 and the second electrode 116 may be electrical conductors. The first electrode 114 and the second electrode 116 may be capable of being coupled to other conductors, permitting the first electrode 114 and the second electrode 116 to receive an electric current or voltage. In some embodiments, the first electrode 114 and the second electrode 116 may be formed from copper. In other embodiments, the first electrode 114 and the second electrode 116 may be formed from other materials, such as aluminum, zinc, silver, alloys of various materials, and conductive polymers having low resistance. In some embodiments, the first electrode 114 and the second electrode 116 may be classified under the American Wire Gauge (AWG) between about 22 AWG and about 30 AWG, having an average diameter between about 0.25 inches and about 0.1 inches. In some embodiments, the first electrode 114 and the second electrode 116 may each be an uninsulated wire loop. In other embodiments, the first electrode 114 and the second electrode 116 may each be electrical contact buttons crimped onto an end of a wire or a bare wire end. In still other embodiments, the first electrode 114 and the second electrode 116 may be components of a circuit board, such as a semiconductor board, disposed in the dressing interface 110. In still other embodiments, the first electrode 114 and the second electrode 116 may each comprise a plurality of electrodes on an electrical circuit.

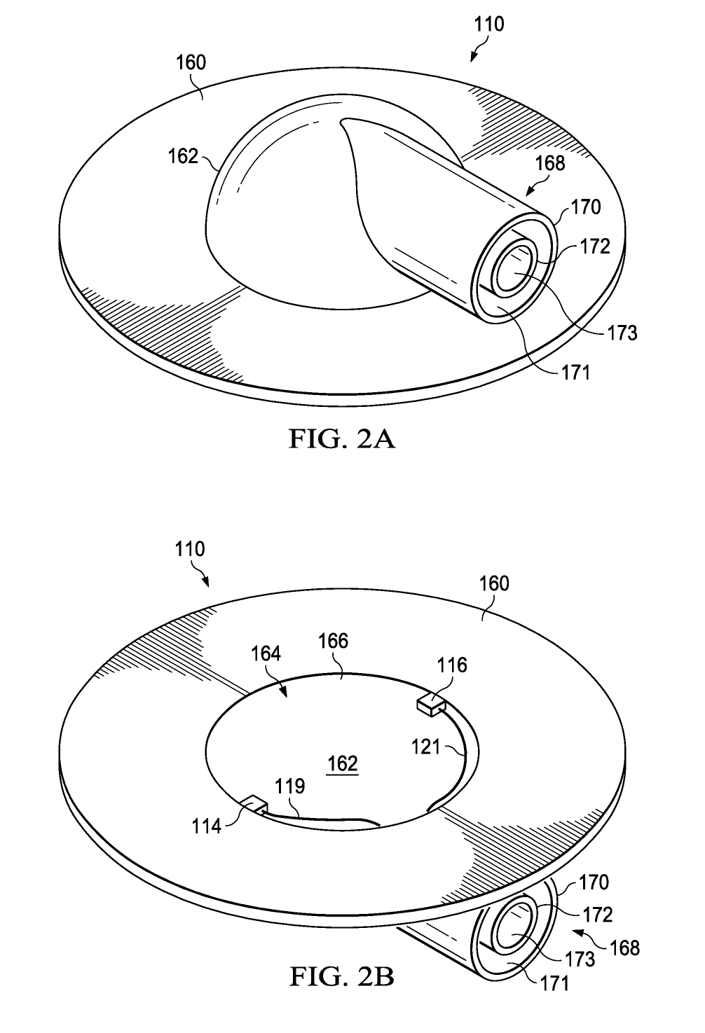

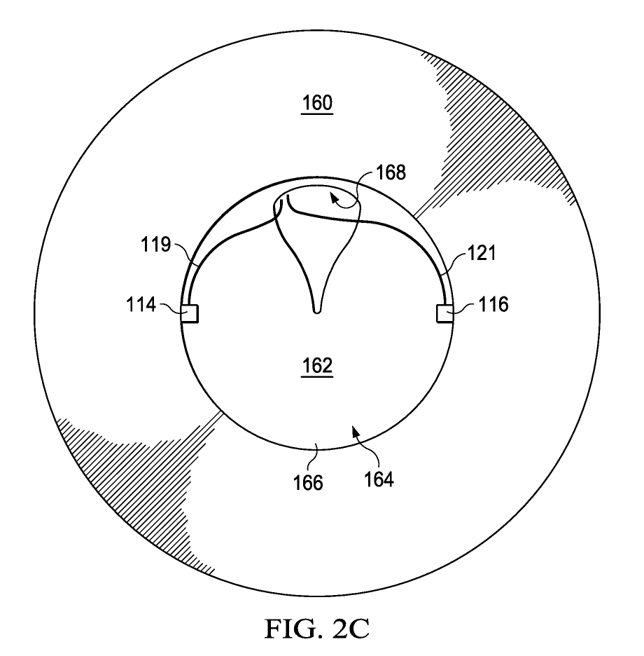

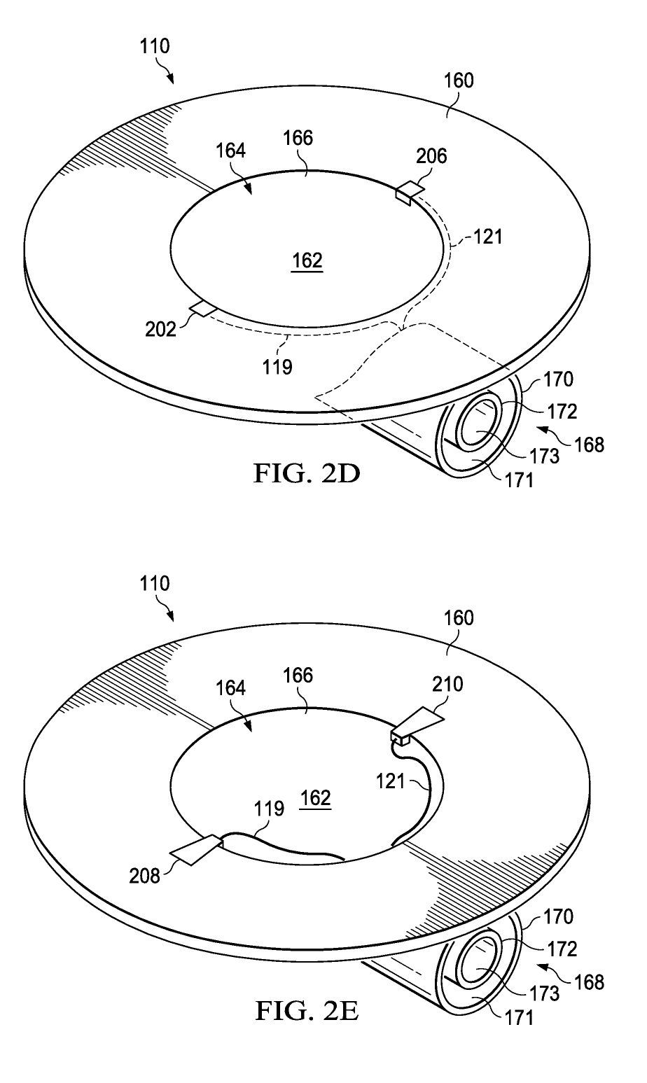

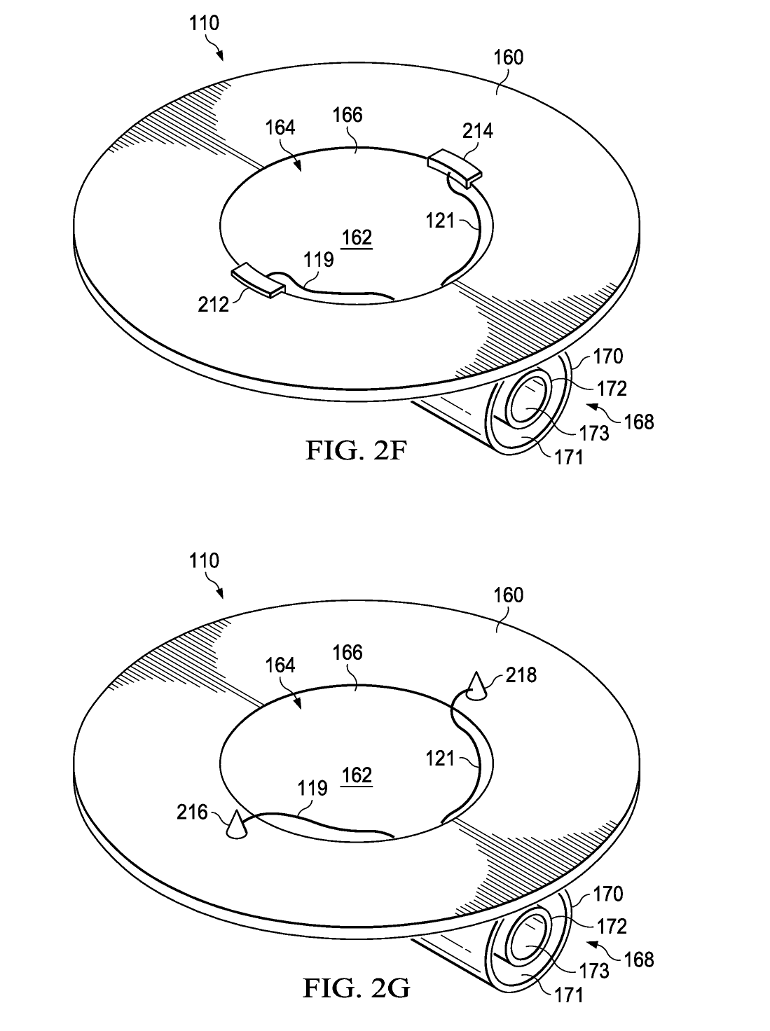

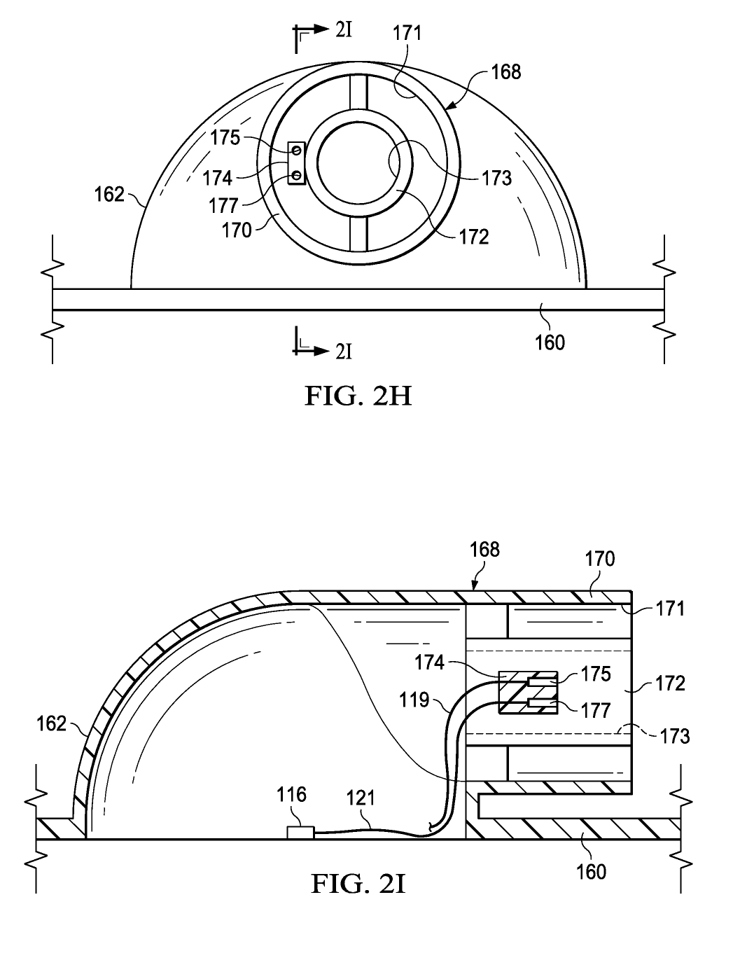

FIG. 2A is a top perspective view of an exemplary embodiment of the dressing interface 110, illustrating additional details that may be associated with some embodiments of the therapy system 100. The dressing interface 110 may include a base 160, a connector body 162, and a conduit port 168. The base 160 may be a disc-shaped body having a diameter at least one order of magnitude larger than a thickness of the base 160. The connector body 162 may be a dome-shaped body coupled to the base 160 and extending away from the base 160 on a first side. In some embodiments, the connector body 162 may form a hemisphere. In other embodiments, the connector body 162 may be less than hemispherical or not be spherical.

The conduit port 168 may be an annular body configured to receive a tube, such as the tube 107. The conduit port 168 may have an annular wall 170 having a first end coupled to the connector body 162. The conduit port 168 may extend away from the connector body 162. In some embodiments, the conduit port 168 may be a multi-lumen port. The annular wall 170 can have an inner diameter that can accommodate the outer diameter of a tube, such as the tube 107. In some embodiments, if the tube 107 is inserted into the annular wall 170, the annular wall 170 may seal to the outer surface of the tube 107. The conduit port 168 also includes an annular wall 172 having a lumen 173. The annular wall 172 may have an outer diameter that is less than an inner diameter of the annular wall 170, forming an annulus 171. In some embodiments, the annulus 171 may be about the thickness of a wall of a tube, such as the tube 107. A tube may be inserted into the annulus 171 so that the annular wall 172 can fit within and be in fluid communication with a primary lumen of the tube.

FIG. 2B is a bottom perspective view of the dressing interface 110 of FIG. 2A, illustrating additional details that may be associated with some embodiments. The base 160 may have an aperture 166 extending through the base 160. In some embodiments, the aperture 166 may be coaxial with the base 160. A diameter of the aperture 166 may be less than a diameter of the base 160. In some embodiments, the base 160 may have attachment device, such as an adhesive. The attachment device may be disposed on a surface of the base 160 proximate to the aperture 166. The attachment device may cover all of the base 160 between the aperture 166 and an outer periphery of the base 160, or the attachment device may cover a portion of the base 160 proximate to the aperture 166. An edge of the connector body 162 may be coupled to the base 160 proximate to the aperture 166. In some embodiments, the edge of the connector body 162 may be coincident with the aperture 166, forming a cavity 164. The cavity 164 may extend from the aperture 166 into the connector body 162. The base 160 may be adjacent at least a portion of the cavity 164. If the dressing interface 110 is positioned at a tissue site, the base 160 may be positioned adjacent the sealing member 106 over the tissue interface 108 so that the cavity 164 is fluidly coupled to the tissue interface 108 through an aperture of the sealing member 106.

The dressing interface 110, including the base 160, the connector body 162, and the conduit port 168, may be made of a semi-rigid material. In a non-limiting example, the dressing interface 110 may be made from a plasticized polyvinyl chloride (PVC), polyurethane, cyclic olefin copolymer elastomer, thermoplastic elastomer, poly acrylic, silicone polymer, or polyether block amide copolymer.

The dressing interface 110 may include the first electrode 114 and the second electrode 116. The first electrode 114 may be disposed in the cavity 164 proximate to the aperture 166 of the base 160. The first electrode 114 may be coupled to a portion of the connector body 162 proximate to the aperture 166. In other embodiments, the first electrode 114 may be coupled to the base 160. In some embodiments, the first electrode 114 may be disposed on a peripheral portion of the aperture 166. The second electrode 116 may also be disposed in the cavity 164 proximate to the aperture 166 of the base 160. The second electrode 116 may be coupled to a portion of the connector body 162. In other embodiments, the second electrode 116 may be coupled to the base 160 proximate to the aperture 166. In some embodiments, the second electrode 116 may be on a peripheral portion of the aperture 166. As shown in FIG. 2B, the first electrode 114 and the second electrode 116 may be on opposite sides of the aperture 166. In other embodiments, the first electrode 114 and the second electrode 116 may be proximate to one another. In still other embodiments, the first electrode 114 and the second electrode 116 may be formed spaced apart, for example on a molded tentacle reaching beyond the perimeter of the base 160. A first electrical conductor, such as a first wire 119, may be coupled to the first electrode 114 to form a portion of the first conductive pathway 113. Similarly, a second electrical conductor, such as a second wire 121, may be coupled to the second electrode 116 to form a first portion of the second conductive pathway 115.

FIG. 2C is a bottom view of the dressing interface 110, illustrating additional details that may be associated with some embodiments. The first wire 119 may be embedded in the connector body 162. The first wire 119 may also be disposed on a surface of the connector body 162 that forms the cavity 164. The first wire 119 may extend from the first electrode 114 to the conduit port 168. The second wire 121 may be embedded in the connector body 162. The second wire 121 may also be disposed on a surface of the connector body 162 that forms the cavity 164. The second wire 121 may extend from the second electrode 116 to the conduit port 168. Both the first wire 119 and the second wire 121 can carry an electrical current. In some embodiments, the first wire 119 and the second wire 121 may be insulated, such as with a polyvinylchloride material. In other embodiments, the first wire 119 and the second wire 121 may be coated with a thin varnish insulation. In still other embodiments, the first wire 119 and the second wire 121 can be bare copper wire embedded in the connector body 162.

As shown in FIG. 2C, the first electrode 114 and the second electrode 116 may be disposed on the dressing interface 110 so that the angle between the first electrode 114 and the second electrode 116 around the aperture 166 is about 180 degrees. In other embodiments, the first electrode 114 and the second electrode 116 may be disposed on the dressing interface 110 so that the angle between the first electrode 114 and the second electrode 116 around the aperture 166 is less than 180 degrees. Preferably, the first electrode 114 and the second electrode 116 are not directly electrically connected to each other. The first electrode 114 and the second electrode 116 may have a collective surface area exposed in the base 160 that comprises a portion of the surface area of the base 160. For example, the first electrode 114 and the second electrode 116 may comprise between about 1% and about 25% of the surface area of the base 160. In some embodiments, a surface of the first electrode 114 and the second electrode 116 exposed in the base 160 may be smooth. In other embodiments, a surface of the first electrode 114 and the second electrode 116 exposed in the base 160 may be textured or rough.

FIG. 2D is a bottom perspective view of the dressing interface 110 of FIG. 2A, illustrating additional details that may be associated with other embodiments. The dressing interface 110 may include the base 160, the connector body 162, the cavity 164, the aperture 166, and the conduit port 168 as previously described. The dressing interface 110 may also include a first electrode 202 and a second electrode 206. The first electrode 202 and the second electrode 206 may be similar to and operate as described above with respect to the first electrode 114 and the second electrode 116. The first electrode 202 and the second electrode 206 may be disposed in the base 160 of the dressing interface 110 adjacent to the aperture 166. Each of the first electrode 202 and the second electrode 206 may have a generally cuboid or square shape. In some embodiments, a surface of each of the first electrode 202 and the second electrode 206 may be curved to match a curvature of the aperture 166 and the cavity 164. In some embodiments, the first electrode 202 and the second electrode 206 may be embedded in the base 160 so that a surface of the first electrode 202 and the second electrode 206 facing away from the base 160 may be flush with a surface of the base 160. In other embodiments, the first electrode 202 and the second electrode 206 may be embedded in the base 160 so that a portion of the first electrode 202 and the second electrode 206 protrude from the base 160. In some embodiments, the first wire 119 and the second wire 121 may be embedded in the connector body 162 so that the first wire 119 and the second wire 121 are covered by the material forming the connector body 162.

FIG. 2E is a bottom perspective view of the dressing interface 110 of FIG. 2A, illustrating additional details that may be associated with other embodiments. The dressing interface 110 may include the base 160, the connector body 162, the cavity 164, the aperture 166, and the conduit port 168 as previously described. The dressing interface 110 may also include a first electrode 208 and a second electrode 210. The first electrode 208 and the second electrode 210 may be similar to and operate as described above with respect to the first electrode 114 and the second electrode 116. The first electrode 208 and the second electrode 210 may be disposed in the base 160 of the dressing interface 110. In some embodiments, the first electrode 208 and the second electrode 210 may each have a trapezoidal shape. The first electrode 208 and the second electrode 210 may each have a first width proximate to the aperture 166 and a second width radially distal from the aperture 166. In some embodiments, the first width may be smaller than the second width so that the first electrode 208 and the second electrode 210 each increase in width extending radially away from the aperture 166. In other embodiments, the first width may be larger than the second width so that the first electrode 208 and the second electrode 210 decrease in width extending radially away from the aperture 166. In some embodiments, a portion of each of the first electrode 208 and the second electrode 210 may protrude from the base 160 into the aperture 166.

FIG. 2F is a bottom perspective view of the dressing interface 110 of FIG. 2A, illustrating additional details that may be associated with other embodiments. The dressing interface 110 may include the base 160, the connector body 162, the cavity 164, the aperture 166, and the conduit port 168 as previously described. The dressing interface 110 may also include a first electrode 212 and a second electrode 214. The first electrode 212 and the second electrode 214 may be similar to and operate as described above with respect to the first electrode 114 and the second electrode 116. The first electrode 212 and the second electrode 214 may be disposed in the base 160 of the dressing interface 110. In some embodiments, the first electrode 212 and the second electrode 214 may be embedded in the base 160 so that a portion of the first electrode 212 and the second electrode 214 protrude from the base 160. In other embodiments, the first electrode 212 and the second electrode 214 may be embedded in the base 160 so that a surface of the first electrode 212 and the second electrode 214 facing away from the base 160 may be flush with a surface of the base 160. The first electrode 212 and the second electrode 214 may each have an arcuate length that is less than a circumference of the aperture 166. The first electrode 212 and the second electrode 214 may each circumscribe at least a portion of the aperture 166.

FIG. 2G is a bottom perspective view of the dressing interface 110 of FIG. 2A, illustrating additional details that may be associated with other embodiments. The dressing interface 110 may include the base 160, the connector body 162, the cavity 164, the aperture 166, and the conduit port 168 as previously described. The dressing interface 110 may also include a first electrode 216 and a second electrode 218. The first electrode 216 and the second electrode 218 may be similar to and operate as described above with respect to the first electrode 114 and the second electrode 116. The first electrode 216 and the second electrode 218 may be disposed in the base 160 of the dressing interface 110. The first electrode 216 and the second electrode 218 may have a generally conical shape having a base and an apex. The base of the first electrode 216 and the second electrode 218 may be coupled to the base 160 proximate to the aperture 166. The first electrode 216 and the second electrode 218 may extend away from a surface of the base 160 to terminate at the apex.

FIG. 2H is an end view of the dressing interface 110, illustrating additional details that may be associated with some embodiments. An electrical connector, such as a receptacle 174, may be disposed within the annulus 171 and coupled to the annular wall 172. For example, the receptacle 174 may be coupled to a side of the annular wall 172. In other embodiments, the receptacle 174 may be coupled to a side of the annular wall 170. The receptacle 174 may be an electrical receptacle having a first slot 175 and a second slot 177. The First slot 175 and the second slot 177 may each be conductive components or conductors configured to receive another conductor to make an electrical connection that permits flow of a current from a first component to a second component through the receptacle 174.

FIG. 2I is a sectional view of the dressing interface 110 taken along line 2I-21 of FIG. 2H, illustrating additional details that may be associated with some embodiments. The annulus 171 may extend from a distal end of the conduit port 168 to the cavity 164. The annulus 171 may be in fluid communication with the cavity 164, permitting fluid communication from an exterior of the dressing interface 110 with the cavity 164. Similarly, the lumen 173 may extend from a distal end of the conduit port 168 to the cavity 164. The lumen 173 may be in fluid communication with the cavity 164, permitting fluid communication from an exterior of the dressing interface 110 with the cavity 164. The receptacle 174 may be electrically connected to the first wire 119 and the second wire 121. For example, the first wire 119 and the second wire 121 may be embedded in the material forming the dressing interface 110. In some embodiments, the first wire 119 and the second wire 121 may terminate at terminals, such as slots or holes, of the receptacle 174, permitting the first wire 119 and the second wire 121 to be electrically connected to other devices through the receptacle 174. In some embodiments, the first wire 119 may terminate at the first slot 175, and the second wire 121 may terminate at the second slot 177.

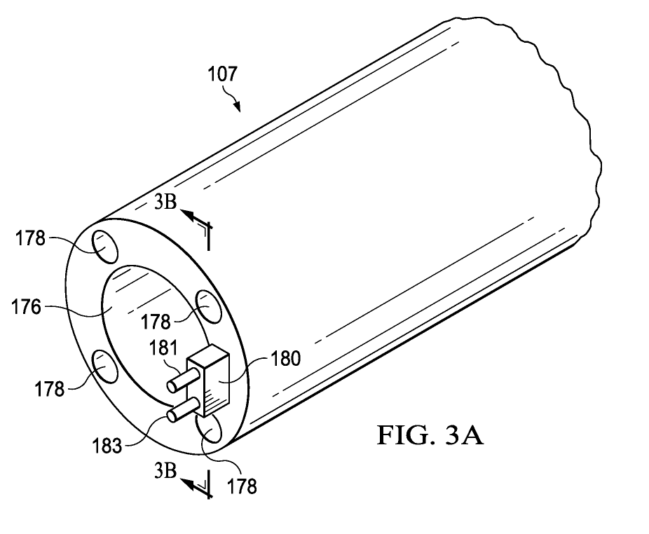

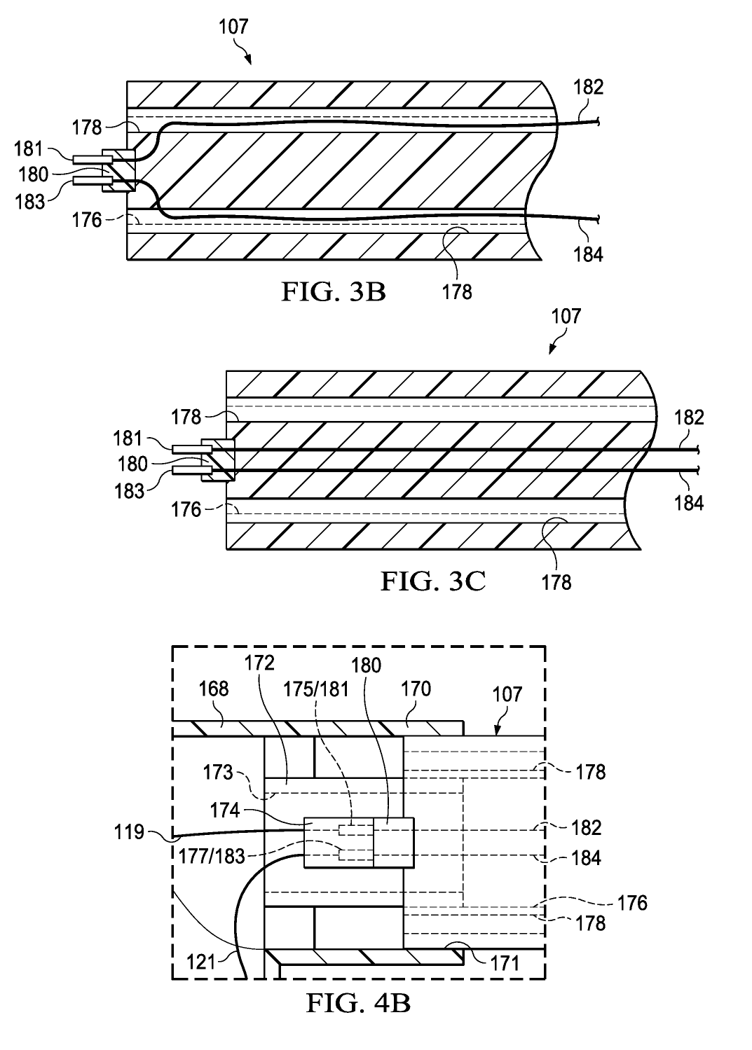

FIG. 3A is a perspective view of the tube 107, illustrating additional details that may be associated with some embodiments of the therapy system 100. The tube 107 may be a multi-lumen tube having a primary lumen 176, and at least one outer lumen 178. Four outer lumens 178 are shown in FIG. 3A; the outer lumens 178 may be separated by a solid portion extending radially around a circumference of the tube 107. In some embodiments, the outer lumens 178 are equidistantly spaced around the tube 107. In other embodiments, there may be more or fewer outer lumens 178 and the spatial arrangement of the primary lumen 176 and the outer lumens 178 may vary. The tube 107 may have a plug 180 disposed on an end of the tube 107. In other embodiments, the plug 180 may be coupled to an exterior of the tube 107. The plug 180 may be an electrical connector that is capable of connecting electrical components. For example, the plug 180 may include a first pin 181 and a second pin 183. The first pin 181 and the second pin 183 may be conductors configured to be inserted into a respective slot of a receptacle to electrically couple to components through the plug 180. In some embodiments, the plug 180 may be configured to mate with the receptacle 174 of the dressing interface 110.

FIG. 3B is a sectional view of the tube 107 of FIG. 3A taken along line 3B-3B, illustrating additional details that may be associated with some embodiments of the therapy system 100 of FIG. 1. A first wire 182 and a second wire 184 may be coupled to the plug 180. For example, the first wire 182 and the second wire 184 may terminate at electrical terminals, such as prongs, blades, or pins, of the plug 180. As shown, the first wire 182 may terminate at the first pin 181, and the second wire 184 may terminate at the second pin 183. The first wire 182, and the second wire 184 may extend from the plug 180 to a corresponding plug on an opposite end of the tube 107. The first wire 182 may form a second portion of the first conductive pathway 113, and the second wire 184 may form a second portion of the second conductive pathway 115. In some embodiments, the first wire 182 and the second wire 184 may be disposed in one or more outer lumens 178. For example, the first wire 182 may be disposed in a first outer lumen 178, and the second wire 184 may be disposed in a second outer lumen 178. In other embodiments, the first wire 182 and the second wire 184 may be disposed in the same outer lumen 178.

FIG. 3C is a sectional view of another embodiment of the tube 107, illustrating additional details that may be associated with some embodiments of the therapy system 100. The tube 107 may be similar to and include the components of the tube described and illustrated with respect to FIG. 3A and FIG. 3B. As shown in FIG. 3C, the first wire 182 and the second wire 184 may be embedded in a wall of the tube, for example, in an annular wall forming the primary lumen 176.

In other embodiments the first conductive pathway 113 and the second conductive pathway 115 may be formed with a separate electrical harness external to the tube 107 or clipped to an exterior of the tube 107. An electronics module could also be positioned at the sealing member 106 and electrically coupled to the first electrode 114 and the second electrode 116. In some embodiments, the electronics module could communicate with the controller wirelessly, or be wired through the first conductive pathway 113 and the second conductive pathway 115.

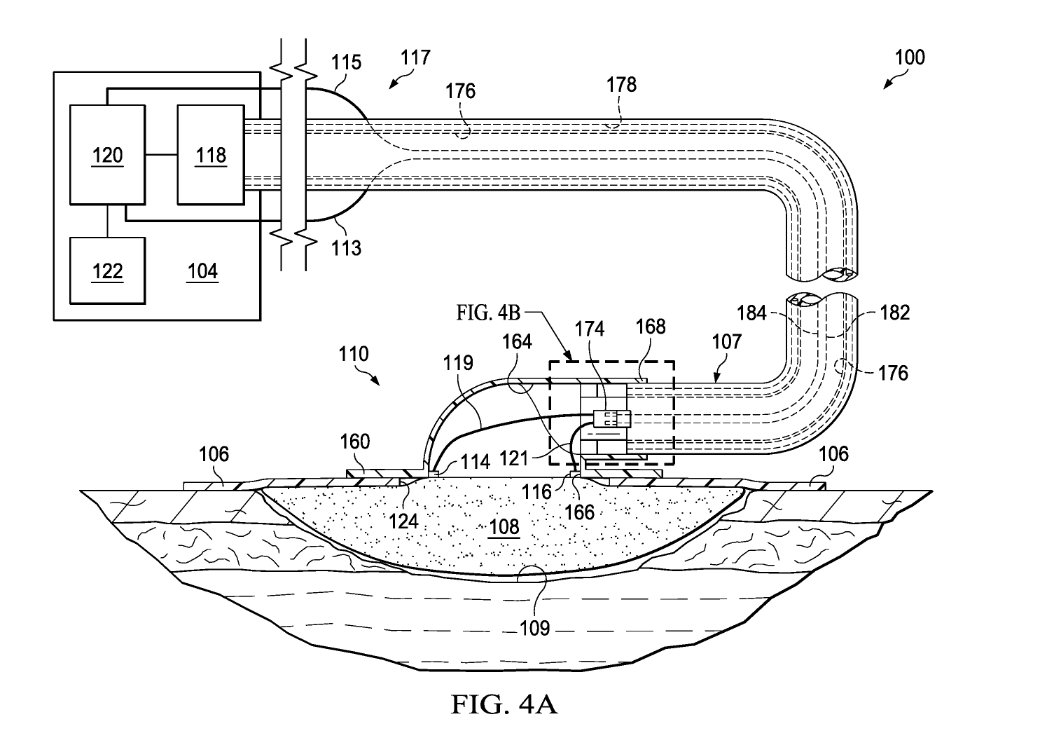

FIG. 4A is a sectional view of the dressing 102 and a schematic view of the therapy system 100 of FIG. 1, illustrating additional details that may be associated with some embodiments. In operation, the tissue interface 108 may be positioned adjacent a tissue site 109. The sealing member 106 can be placed over the tissue interface 108 and the tissue site 109 and sealed to tissue surrounding the tissue site 109 to create a sealed therapeutic environment. An aperture 124 may be formed in the sealing member 106. In some embodiments, the aperture 124 may be formed prior to placement of the sealing member 106 over the tissue site 109. In other embodiments, the aperture 124 may be formed after placement of the sealing member 106 over the tissue site 109. The dressing interface 110 may be positioned over the sealing member 106 so that the aperture 166 of the dressing interface 110 is generally aligned with the aperture 124 of the sealing member 106. In other embodiments, the dressing interface 110 may be coupled to the sealing member 106 by bonding, adhering, welding, or another joining method. The dressing interface 110 may be sealed to the sealing member 106 so that the aperture 166 is in fluid communication with the aperture 124. The attachment device coupled to the base 160 of the dressing interface 110 may adhere the dressing interface 110 to the sealing member 106, providing a fluid seal between the dressing interface 110 and the sealing member 106. In some embodiments, the aperture 124 may be larger than the aperture 166 so that a portion of the base 160 may contact the tissue interface 108 through the aperture 124. If the dressing interface 110 is sealed to the sealing member 106, the first electrode 114 and the second electrode 116 may contact the tissue interface 108. In some embodiments, the first electrode 114 and the second electrode 116 may contact the conductive portion of the tissue interface 108. For example, the tissue interface 108 may be coated with silver, and the first electrode 114 and the second electrode 116 may contact the silver coating of the tissue interface 108. The tissue interface 108 may be in fluid communication with the aperture 124 of the sealing member 106 and the aperture 166 of the dressing interface 110. Similarly, the lumen 173 and the annulus 171 may be in fluid communication with the cavity 164, and the cavity 164 may be in fluid communication with the tissue interface 108 through the aperture 166 and the aperture 124.

In other embodiments, the dressing interface 110 may be adhered directly to the tissue interface 108. The sealing member 106 may be positioned over the tissue interface 108 and the dressing interface 110, and sealed to tissue surrounding the tissue site 109, the tissue interface 108, and a surface of the base 160 of the dressing interface 110. The aperture 124 may be formed so that the connector body 162 may pass through the aperture 124.

A first end of the tube 107 may be inserted into the conduit port 168 to fluidly couple and electrically couple the dressing 102 to the tube 107. A second end of the tube 107 may be coupled to the negative-pressure source 104 to fluidly couple and electrically couple the tube 107 to the negative-pressure source 104. The primary lumen 176 may be in fluid communication with the pump 118, and the outer lumens 178 may be terminated and sealed at the negative-pressure source 104. In some embodiments, the first wire 182 may be electrically coupled to the controller 120 to complete the first conductive pathway 113, and the second wire 184 may be electrically coupled to the controller 120 to complete the second conductive pathway 115.

FIG. 4B is a sectional view of a portion of the dressing interface 110 and the tube 107, illustrating additional details that may be associated with some embodiments. A primary lumen 176 of the tube 107 may be in fluid communication with the lumen 173. Similarly, the outer lumens 178 may be in fluid communication with the annulus 171. An outer surface of the tube 107 may engage in an interference fit with the annular wall 170 so that the tube 107 is sealed to the conduit port 168. Similarly, an inner surface of the primary lumen 176 may engage in an interference fit with an outer surface of the annular wall 172, sealing the primary lumen 176 to the annular wall 172. Both the primary lumen 176 and the outer lumens 178 may be in fluid communication with the sealed therapeutic environment through the aperture 124 in the sealing member 106, the aperture 166 in the dressing interface 110, and the cavity 164 of the dressing interface 110. If the tube 107 is inserted into the conduit port 168, the plug 180 may be aligned with the receptacle 174. The receptacle 174 may receive the plug 180 so that the first pin 181 is inserted into the first slot 175 and the second pin 183 is inserted into the second slot 177.

In operation, the controller 120 may include a power source that provides a constant current to the sensing circuit 117 that induces a voltage drop across the conductive portion of the tissue interface 108 between the first electrode 114 and the second electrode 116, i.e., a load voltage. In some embodiments, the power source may provide a constant current of about 0.1 milliamps, wherein the load voltage may vary from about 0.2 volts to about 1.0 volts. In other embodiments, the constant current may be less than about 0.3 milliamps. In other embodiments, the constant current may be in a range being applied to the conductive portion of the tissue interface that meets the IEC601-1, UL2601-1 Safety Standards for medical devices. The controller 120 may be adapted to determine an initial load voltage and/or an initial resistance of the conductive portion of the tissue interface 108 before negative pressure is applied to the therapeutic environment. The controller 120 may correlate the initial load voltage measured to an ambient pressure because a negative pressure has not yet been applied to the sealed therapeutic environment.

In other embodiments, the controller 120 may apply a non-constant current to the sensing circuit 117 from an alternating-current power source. The supplied current could be sinusoidal, and an electrical impedance across the tissue interface 108 could be determined by the controller 120. The supplied current could be provided at different frequencies and the impedance could be measured at each frequency supplied.

The controller 120 may actuate the pump 118 to begin applying negative pressure to the therapeutic environment and draw fluids from the therapeutic environment through the tissue interface 108, the dressing interface 110, and the tube 107. The tissue interface 108 deforms or compresses if negative pressure is applied to the therapeutic environment causing fluids to be withdrawn from the therapeutic environment. If the tissue interface 108 comprises foam having a conductive portion, as the tissue interface 108 compresses, the resistance of the conductive portion decreases, causing the load voltage to decrease proportionally. The decrease in the load voltage can be correlated to a decrease in pressure as a measure of the negative pressure at the tissue site. The change in the load voltage can occur as the tissue interface 108 is compressed by an increasing negative pressure in the sealed therapeutic environment. In one example embodiment, conductive components of the tissue interface 108 may be drawn closer together if the tissue interface 108 is compressed, thereby reducing the resistance, or increasing the conductivity, of the tissue interface 108. As the conductive components of the tissue interface 108 draw toward one another, additional conductive pathways between the first electrode 114 and the second electrode 116 can be created. An increase in the conductive pathways provides more paths for the applied constant current to flow between the first electrode 114 and the second electrode 116, causing an increase in conductivity. The controller 120 may measure the load voltage across the conductive portion of the tissue interface 108 as described above to determine the pressure at the tissue site. If the load voltage approaches a target voltage (TV) corresponding to a target pressure (TP) desired for therapeutic treatment, the controller 120 may stop the pump 118, determining that the desired therapeutic pressure is being applied to the tissue site. For example, the target voltage (TV) may be correlated with a target pressure (TP) of about 120 mm Hg. The target voltage (TV) and target pressure (TP) may be a fixed value (either maximum or minimum values), intermittent values switching between two specific values including a value associated with no negative pressure being applied (e.g., ambient pressure), or variable values defined by various shapes, such as sinusoidal, saw-tooth, triangular, and other dynamic pressure type therapies.

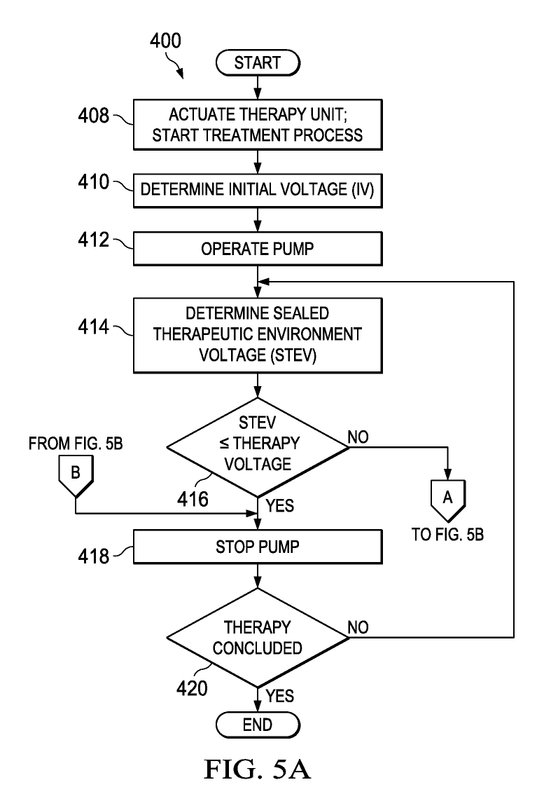

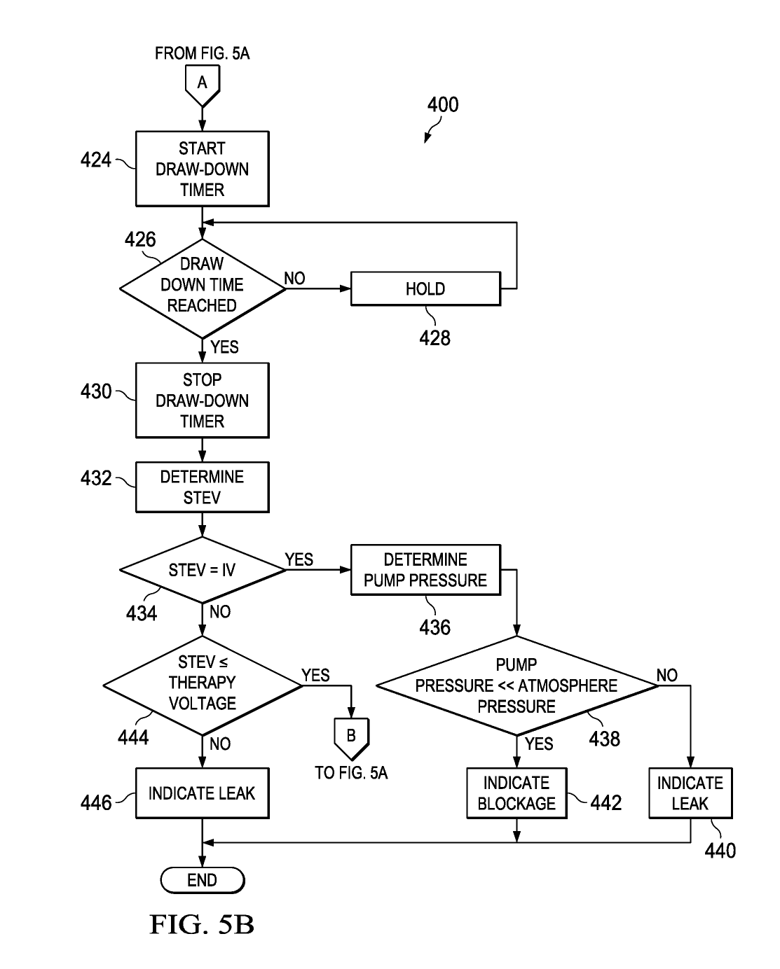

FIG. 5A and FIG. 5B illustrate a flow chart 400 depicting logical operations that can be implemented in some embodiments of the therapy system 100 of FIG. 4A and FIG. 4B during the provision of negative-pressure therapy. For example, the operations may be implemented by a controller, such as the controller 120, configured to execute the operations to complete a process. The dressing 102 may be applied to the tissue site 109, and the dressing interface 110 may be coupled to the dressing 102. The tube 107 may be coupled to the dressing interface 110 and the negative-pressure source 104, as described above with respect to FIG. 4A and FIG. 4B.

As depicted in FIG. 5A and FIG. 5B, at block 408, the process actuates a therapy unit and starts a treatment process. For example, the negative-pressure source 104 may be turned on and a treatment, such as negative-pressure therapy or fluid instillation therapy, may be programmed with the input-output device 122. In some embodiments, after selecting negative-pressure therapy at block 408, a user may program a total number of negative-pressure therapy cycles included in the treatment process. A negative-pressure cycle is a period where a pressure in the sealed therapeutic environment is about the therapy pressure, followed by a period where a pressure in the sealed therapeutic environment is about the ambient pressure. In other embodiments, after selecting negative-pressure therapy at block 408, a user may enter a total time period for negative-pressure therapy. For example, a user may select to conduct negative-pressure therapy for about 2 hours. In response, the controller 120 can actuate a timer at the outset of negative-pressure therapy. The controller 120 may also be initially programmed by a caregiver or user to provide a specific target pressure (TP). The target pressure (TP) corresponds to a specific target voltage (TV) as described above, wherein the target voltage (TV) may also be referred to herein as the therapy voltage.

In some embodiments, a range of target voltages (TV) can be specified in a look-up table stored in memory component of the controller 120. For example, a user can program the controller 120 using the input-output device 122, specifying the material of the tissue interface 108 and the target pressure (TP). In response, the controller 120 can access a look-up table for the selected material having values corresponding to the target voltage (TV) for the material of the tissue interface 108 and its conductive portion corresponding to the specific target pressure (TP). A secondary look-up table may contain information associated with other conditions of therapy. In an exemplary embodiment, a look-up table for the tissue interface 108 formed from GranuFoam Silver® of Kinetic Concepts, Inc. of San Antonio, Tex., for a power source supplying about 0.1 mA and in dry conditions. In the example, the dry condition is specified in a secondary look-up table. A target voltage for a target pressure of 0 mm Hg is about 1V. At a target pressure of about 50 mm Hg, the corresponding target voltage is about 0.7V. At a target pressure of about 100 mm Hg, the corresponding target voltage is about 0.8V. At a target pressure of about 125 mm Hg, the corresponding target voltage is about 0.85V. At a target pressure of about 150 mm Hg, the corresponding target voltage is about 0.89V. At a target pressure of about 200 mm Hg, the corresponding target voltage is about 0.91V. For a similar tissue interface 108 with a similar power source where the tissue interface 108 is disposed in a saline solution (i.e., a wet state), at a target pressure of about 0 mm Hg, the corresponding target voltage is about 1.0V. At a target pressure of about 125 mm Hg, the corresponding target voltage is about 0.8V. At a target pressure of about 200 mm Hg, the corresponding target voltage is about 0.68V.

At block 410, the process determines an initial load voltage IV. For example, the controller 120 may use the power source to supply a constant current to the sensing circuit 117, inducing the initial load voltage across the tissue interface 108. The controller 120 may assign the load voltage measured across the tissue interface 108 as the initial load voltage IV. Therapy may be applied to the tissue site by operating the pump at block 412. For example, the controller 120 may actuate the pump 118, causing the pump 118 to draw fluid from the sealed therapeutic environment through the dressing 102 and the tube 107. At block 414, the process may determine a sealed therapeutic environment voltage STEV. For example, during operation of the pump 118, the controller 120 may actuate the power supply to supply a constant current to sensing circuit 117, inducing a load voltage across the tissue interface 108. The load voltage as the pump 118 draws fluid from the sealed therapeutic environment may be stored by the controller 120 as the sealed therapeutic environment voltage STEV.

At block 416, the process compares the sealed therapeutic environment voltage STEV to the therapy voltage. For example, the controller 120 may compare the sealed therapeutic environment voltage STEV to the therapy voltage associated with the target pressure programmed by a user. If the sealed therapeutic environment voltage STEV is less than or equal to the therapy voltage, the process continues on the YES path to block 418, where the process stops the pump. For example, if the sealed therapeutic environment voltage STEV is less than or equal to the therapy voltage, the load voltage across the tissue interface 108 is less than or equal to the load voltage across the tissue interface 108 that is correlated to the target pressure in the sealed therapeutic environment. In response, the controller 120 determines that a pressure in the sealed therapeutic environment is less than or equal to the target pressure (TP), and the controller 120 stops operation of the pump 118.

The process can determine if therapy has concluded at block 420. For example, the controller 120 can determine if negative-pressure therapy has concluded. The controller 120 may determine if the appropriate number of negative-pressure cycles has occurred. The controller 120 can also compare the value of the timer initiated at block 408 to the total time period for negative-pressure therapy entered in the input-output device 122. If therapy has concluded, the process follows the YES path, where the process ends.

At block 420, if therapy is not concluded, the process follows the NO path to block 414, where the process continues. For example, if the value of the timer initiated at block 408 is less than the total time period for therapy, also entered at block 408, the controller 120 can determine that therapy has not concluded. The controller 120 can also determine that the appropriate number of negative-pressure cycles has not occurred.

At block 414, the process determines the sealed therapeutic environment voltage STEV. At block 416, the sealed therapeutic environment voltage STEV is compared to the therapy voltage. If the sealed therapeutic environment voltage STEV is greater than the therapy voltage, the process follows the NO path to block 424. At block 424, the process starts a draw-down timer. For example, the controller 120 may start another timer. At block 426, the process determines if a draw down time has been reached. A draw down time may be the expected time for a pressure in the sealed therapeutic environment to reach the therapy pressure. The controller 120 can monitor the drawn-down timer started at block 424 to determine if the draw down time is reached.

At block 426, if the draw down time is not reached, the process continues on the NO path to block 428. At block 428, the process holds for a predetermined period of time. For example, the controller 120 may continue operation of the pump 118 for a period of 30 seconds. The process then returns to block 426. At block 426, if the draw down time is reached, the process continues on the YES path to block 430. At block 430, the process stops the draw-down timer. For example, the controller 120 can stop the draw-down timer. In some embodiments, the controller 120 may reset the draw-down timer at block 430.

At block 432, the process determines the sealed therapeutic environment voltage STEV. For example, the controller 120 actuates the power supply to supply the constant current to the sensing circuit 117, inducing a load voltage across the tissue interface 108. The controller 120 can store the induced load voltage as the sealed therapeutic environment voltage STEV. At block 434, the process compares the sealed therapeutic environment voltage STEV to the initial load voltage IV. If the sealed therapeutic environment voltage STEV is about the initial load voltage IV, the process continues on the YES path to block 436. At block 436, the process determines a pump pressure. For example, the controller 120 determines a pressure at a negative-pressure outlet of the pump 118. The negative-pressure source 104 can include a sensor coupled to the negative-pressure outlet of the pump 118. The sensor may be electrically coupled to the controller 120 to determine a pressure at the negative-pressure outlet of the pump 118. The controller 120 can also determine an electrical load of the pump 118 to determine a pump pressure. The electrical load of the pump 118 may be a value associated with the electrical draw of the pump 118 to maintain for operation of the pump 118. The electrical load of the pump 118 may be associated with an expected pressure at the pump outlet.

At block 438, the process compares the pump pressure to an atmospheric pressure. For example, the controller 120 compares the pump pressure at the negative-pressure outlet of the pump 118 to an atmospheric pressure surrounding the negative-pressure source 104. If the pump pressure is not much less than the atmospheric pressure, the process follows the NO path to block 440. At block 440, the process indicates a leak and the process ends. For example, the controller 120 can indicate a leak through the input-output device 122, and the process ends. At block 438, if the pressure at the negative-pressure outlet of the pump 118 is much less than the atmospheric pressure, the process follows the YES path to block 442. At block 442, the process indicates a blockage and the process ends. For example, the controller 120 can indicate a blockage condition through the input-output device 122.

At block 434, if the sealed therapeutic environment voltage STEV is not about the initial voltage IV, the process follows the NO path to block 444. At block 444, the process determines if the sealed therapeutic environment voltage STEV is less than or equal to the therapy voltage. For example, the controller 120 determines if the sealed therapeutic environment voltage STEV is less than or equal to the therapy voltage. If the sealed therapeutic environment voltage STEV is less than or equal to the therapy voltage, the process continues to block 420 and continues as previously described. If the sealed therapeutic environment voltage STEV is greater than the therapy voltage, the process continues to block 446. At block 446, the process indicates a leak. For example, the controller 120 indicates a leak on the input-output device 122.

As described herein, the controller 120 may operate the therapy system 100 to provide on-off control of therapy at a tissue site. In other embodiments, the controller 120 may be programmed to provide variable control of therapy, control of instillation therapy, or to operate the pump 118 at variable frequencies in response to pressure determined through use of the sensing circuit 117.

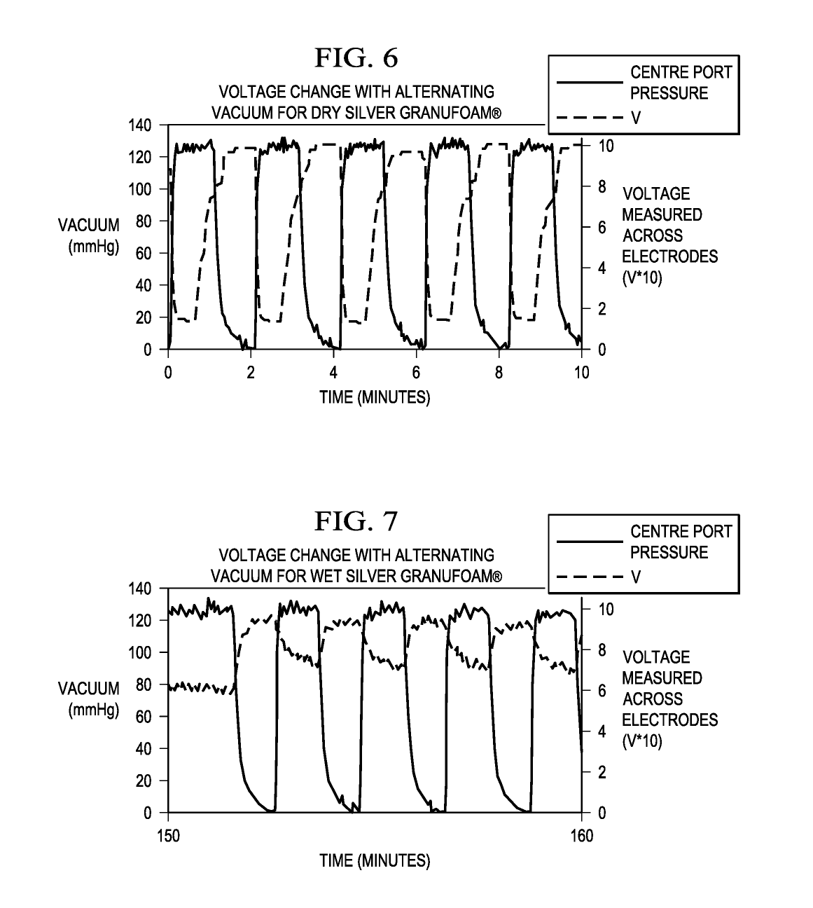

FIG. 6 and FIG. 7 are graphical depictions of voltages measured across a tissue interface in an example of the therapy system 100. In the example, a tissue interface formed from a GranuFoam Silver® of Kinetic Concepts, Inc. of San Antonio, Tex. was placed on a substrate material and covered with a sealing member to form a sealed space containing the tissue interface. A dressing interface, such as a SensaT.R.A.C.® pad of Kinetic Concepts, Inc., of San Antonio, Tex. was modified so that two electrodes were supported by a base of the dressing interface. An opening was formed in the sealing member and the dressing interface was positioned over the opening so that the two electrodes contacted the tissue interface. An alternating pressure was applied to the tissue interface and a small constant current, about 0.1 milliamperes (mA), was applied across the electrodes. The voltage across the electrodes was measured both in a dry state (FIG. 6), and a wet state (FIG. 7).

As illustrated in FIG. 6, the voltage measured across the tissue interface changed as the negative-pressure in the sealed space changed. For example, negative pressure ranging between 0 mm Hg and 120 mmHg was supplied to the tissue interface in the sealed space. During each cycle, a negative-pressure was supplied for about 1 minute and removed for about 1 minute. As the negative-pressure was supplied, the voltage measured decreased from about 1.0 V to about 0.2 V. As fluid was drawn from the sealed space, the voltage measured changed in time with the change in negative pressure in the sealed space. For example, as the negative pressure increased from 0 mm Hg to about 120 mm Hg, the voltage measured decreased from 1.0 V to about 0.2 V, and the change in pressure and the change in voltage occurred in about the same amount of time. Similarly, as the pump ceased operation, the voltage measured began to increase, returning to about 1.0 V as the sealed space was vented to the atmosphere. As shown in FIG. 6, five negative-pressure therapy cycles were measured, each showing a similar correlation between the pressure change and the change in the measured voltage. The change in voltage measured indicates that as negative pressure increased, the conductivity of the tissue interface also increased.

A similar correlation occurs if the tissue interface is wet. In another example, the tissue interface was instilled with a saline content of 0.9% (approximately 9 grams NaCl for about 1000 ml H₂O). Fluid was drawn from the sealed space, causing the negative pressure to increase from about 0 mm Hg to about 130 mmHg. As the negative-pressure increased, the voltage measured across the tissue interface decreased from about 1.0 V to about 0.7 V. As fluid was drawn from the sealed space, the voltage measured changed in time with the change in negative pressure in the sealed space. For example, as the negative pressure increased from 0 mm Hg to about 130 mm Hg, the voltage measured decreased from 1.0 V to about 0.7 V and the change in pressure and the change in voltage occurred in about the same amount of time. Similarly, as the pump ceased operation, the voltage measured began to increase, returning to about 1.0 V as the sealed space was vented to the atmosphere. As shown in FIG. 7, five negative-pressure therapy cycles were measured, each showing a similar correlation between the pressure change and the change in the measured voltage. The change in voltage measured indicates that as negative pressure increased, the conductivity of the tissue interface also increased.

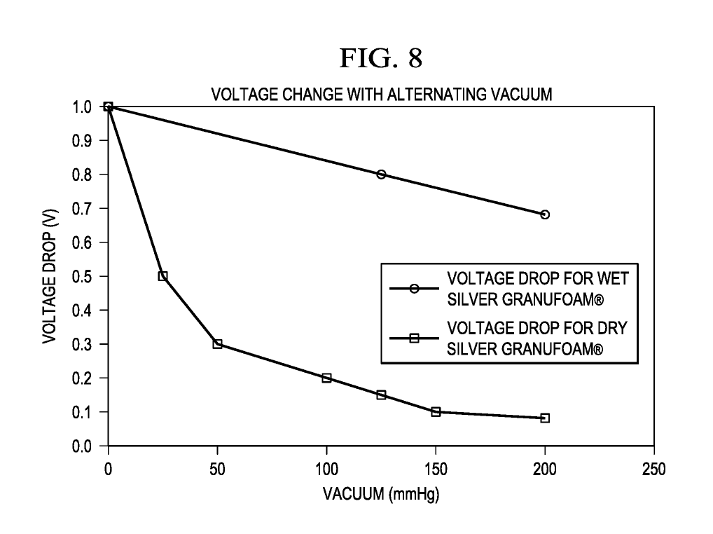

FIG. 8 is another graphical depiction of voltages measured across a tissue interface in an example of the therapy system 100. In the example, a tissue interface formed from a GranuFoam Silver® of Kinetic Concepts, Inc. of San Antonio, Tex. was placed on a substrate material and covered with a sealing member to form a sealed space containing the tissue interface. A dressing interface, such as a SensaT.R.A.C.® pad of Kinetic Concepts, Inc., of San Antonio, Tex. was modified so that two electrodes were supported by a base of the dressing interface. An opening was formed in the sealing member and the dressing interface was positioned over the opening so that the two electrodes contacted the tissue interface. An alternating pressure was applied to the tissue interface and a small constant current, about 0.1 milliamperes (mA), was applied across the electrodes. The voltage across the electrodes was measured both in a dry state and a wet state.

The voltage measured across the tissue interface changed as the negative-pressure in the sealed space changed. For example, negative pressure ranging between 0 mm Hg and 120 mmHg was supplied to the tissue interface in the sealed space. For example, at a negative pressure of about 0 mm Hg, the voltage measured across the tissue interface was about 1V. At a negative pressure of about 25 mm Hg, the voltage measured across the tissue interface was about 0.5V. At a negative pressure of about 50 mm Hg, the voltage measured across the tissue interface was about 0.3V. At a negative pressure of about 100 mm Hg, the voltage measured across the tissue interface was about 0.2V. At a negative pressure of about 125 mm Hg, the voltage measured across the tissue interface was about 0.15V. At a negative pressure of about 150 mm Hg, the voltage measured across the tissue interface was about 0.1V. At a negative pressure of about 200 mm Hg, the voltage measured across the tissue interface was about 0.09V. The change in voltage measured indicates that as negative pressure increased, the conductivity of the tissue interface also increased.

A similar correlation occurs if the tissue interface is wet. In another example, the tissue interface was instilled with a saline content of 0.9% (approximately 9 grams NaCl for about 1000 ml H₂O). Fluid was drawn from the sealed space, causing the negative pressure to increase from about 0 mm Hg to about 130 mmHg. For example, at a negative pressure of about 0 mm Hg, the voltage measured across the tissue interface was about 1.0V. At a negative pressure of about 125 mm Hg, the voltage measured across the tissue interface was about 0.8V. At a negative pressure of about 200 mm Hg, the voltage measured across the tissue interface was about 0.68V. The change in voltage measured indicates that as negative pressure increased, the conductivity of the tissue interface also increased.

The systems, apparatuses, and methods described herein may provide significant advantages. For example, the system does not require fluid flow through outer or peripheral lumens. Consequently, an occlusion or a blockage does not prevent the system from determining a pressure at a tissue site. The pressuring sensing system also works well in high flow situations where pressure may fluctuate rapidly. In other pressure sensing systems, rapid fluctuation caused by highly exudating tissue sites can increase the likelihood of fluid causing a blockage or occlusion that would inhibit accurate pressure sensing. The system described herein provides a pressure-sensing mechanism that is capable of pressure sensing regardless of wound conditions, and can do so with fewer alarms.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A system for determining a pressure in a sealed therapeutic environment provided by a dressing, the system comprising:
a tissue interface configured to be positioned adjacent a tissue site, at least a portion of the tissue interface being electrically conductive;
a sealing member configured to be disposed over the tissue interface to form the sealed therapeutic environment; and
a dressing interface configured to fluidly connect the sealed therapeutic environment with a therapy unit, the dressing interface further configured to be electrically coupled to the tissue interface and the therapy unit.

2.-4. (canceled)

5. The system of claim 1, wherein the tissue interface comprises a polyurethane foam having a conductive coating.

6. The system of claim 1, wherein the tissue interface comprises a poly aniline foam.

7. The system of claim 1, wherein the tissue interface comprises a poly acetylene foam.

8. The system of claim 1, wherein the tissue interface comprises a poly polystyrene sulphonate foam.

9. (canceled)

10. The system of claim 1, further comprising a conductive material coating the tissue interface.

11.-14. (canceled)

15. The system of claim 1, wherein the dressing interface comprises:
a base having an aperture;
a conduit port configured to receive a tube having at least one lumen;
a connector body having a cavity fluidly coupling the aperture to the conduit port; and
two electrodes coupled to the base, the two electrodes separated from each other and configured to be electrically coupled to the therapy unit.

16.-23. (canceled)

24. A system for treating a tissue site comprising:
a manifold having a conductive portion capable of conducting an electric current and configured to be positioned adjacent to the tissue site;
a cover configured to be positioned over the manifold and the tissue site to form a therapeutic environment;
a connector configured to be fluidly coupled to the therapeutic environment and the manifold to a source of negative pressure;
two electrodes coupled to the connector and configured to be electrically coupled to the conductive portion of the manifold; and
a therapy unit configured to fluidly couple the source of negative pressure to the connector and electrically coupled to the two electrodes to provide a current to the conductive portion of the manifold, the therapy unit further configured to measure a therapy voltage across the two electrodes as a measure of a therapy pressure within the therapeutic environment in response to pressure being provided by the source of negative pressure to the therapeutic environment and the manifold.

25. (canceled)

26. (canceled)

27. The system of claim 24, wherein the manifold comprises a polyurethane foam and the conductive portion comprises a conductive coating.

28.-31. (canceled)

32. The system of claim 24, wherein the conductive portion comprises a conductive material coating the manifold.

33. The system of claim 32, wherein the conductive material is silver.

34. The system of claim 32, wherein the conductive material is copper.

35. The system of claim 32, wherein the conductive material is a metal.

36. The system of claim 24, wherein the therapy voltage decreases as the therapy pressure decreases.

37. The system of claim 24, wherein the therapy unit computes the therapy pressure based on the therapy voltage being measured.

38. The system of claim 24, wherein the connector comprises:
a base having an aperture;
a conduit port configured to receive a tube having at least one lumen;
a connector body having a cavity fluidly coupling the aperture to the conduit port; and
the electrodes are coupled to the base and separated from each other, the electrodes configured to be electrically coupled to the therapy unit.

39. (canceled)

40. A method for determining a pressure in a sealed therapeutic environment, the method comprising:
determining an initial voltage across at least two electrodes electrically connected to a tissue interface disposed in the sealed therapeutic environment;
determining a sealed therapeutic environment voltage after operation of a therapy unit;
if the sealed therapeutic environment voltage is about the same as a therapy voltage, identifying the pressure in the sealed therapeutic environment as a therapy pressure; and
if the sealed therapeutic environment voltage is not about the same as a therapy voltage, identifying the pressure in the sealed therapeutic environment is not about the therapy pressure.

41. The method of claim 40, wherein if the sealed therapeutic environment voltage is about the initial voltage, the method further comprises:
starting a timer;
determining if a draw down time is reached;
if the draw down time is not reached, holding; and
if the draw down time is reached, stopping the timer.

42. The method of claim 41, wherein if the draw down time is reached, the method further comprises:
determining the sealed therapeutic environment voltage;
if the sealed therapeutic environment voltage is about the therapy voltage, identifying the pressure in the sealed therapeutic environment as the therapy pressure; and
if the sealed therapeutic environment voltage is not about the therapy voltage, indicating a leak.

43. The method of claim 41, wherein if the draw down time is reached, the method further comprises:
determining the sealed therapeutic environment voltage;
if the sealed therapeutic environment voltage is about the initial voltage, indicating a blockage; and
if the sealed therapeutic environment voltage is not about the initial voltage, determining if the sealed therapeutic environment voltage is about the therapy voltage;
if the sealed therapeutic environment voltage is about the therapy voltage, identifying the pressure in the sealed therapeutic environment as the therapy pressure; and
if the sealed therapeutic environment voltage is not about the therapy voltage, indicating a leak.

44. (canceled)
